(19) United States
(12) Patent Application Publication
(10) Pub. No.: US 2006/0287204 A1
(43) Pub. Date: Dec. 21, 2006

(54) Cyclopropanecarbonitriles and their use in perfume compositions

(76) Inventors: Anubhav P.S. Narula, Hazlet, NJ (US); Edward Mark Arruda, Cliffwood, NJ (US); Adam Jan Janczuk, Parlin, NJ (US); Franc T. Schiet, Naarden (NL)

Correspondence Address: INTERNATIONAL FLAVORS & FRAGRANCES INC., 521 WEST 57TH ST, NEW YORK, NY, 10019, US

(21) Appl. No.: 11/154,399

(22) Filed: Jun. 16, 2005

(51) Int. Cl. C07C 255/45; C11D 3/50; A61K 8/40

(52) U.S. Cl. 510/101; 558/434; 512/8

(57) ABSTRACT

The present invention is directed to novel cyclopropanecarbonitrile compounds of the general formula 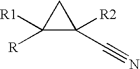 wherein R is a hydrogen, or a straight, branched, or cyclic hydrocarbon moiety consisting of 1 to 30 carbon atoms and containing single and/or double bonds; R1 is a hydrogen or a straight, branched, or cyclic hydrocarbon moiety consisting of 1 to 30 carbon atoms and containing single and/or double bonds and R2 is a hydrogen or a straight, branched, or cyclic hydrocarbon moiety consisting of 1 to 30 carbon atoms and containing single and/or double bonds.

### FIELD OF THE INVENTION

The present invention relates to new chemical entities and the incorporation and use of the new chemical entities as fragrance materials.

### BACKGROUND OF THE INVENTION

There is an ongoing need in the fragrance industry to provide new chemicals to give perfumers and other persons ability to create new fragrances for perfumes, colognes and personal care products. Those with skill in the art appreciate how differences in the chemical structure of the molecule can result in significant differences in the odor, notes and characteristics of a molecule. These variations and the ongoing need to discover and use the new chemicals in the development of new fragrances allows perfumers to apply the new compounds in creating new fragrances.

### SUMMARY OF THE INVENTION

The present invention provides novel chemicals, and the use of the chemicals to enhance the fragrance of perfumes, toilet waters, colognes, personal products and the like. In addition, the present invention is directed to the use of the novel chemicals to enhance fragrance in perfumes, toilet waters, colognes, personal products and the like.

More specifically, the present invention is directed to the novel cyclopropanecarbonitrile compounds, represented by the general structure of Formula 1 set forth below: 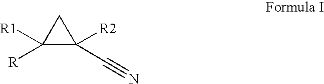
wherein R is a hydrogen, or a straight, branched, or cyclic hydrocarbon moiety consisting of 1 to 30 carbon atoms and containing single and/or double bonds; R1 is a hydrogen or a straight, branched, or cyclic hydrocarbon moiety consisting of 1 to 30 carbon atoms and containing single and/or double bonds and R2 is a hydrogen or a straight, branched, or cyclic hydrocarbon moiety consisting of 1 to 30 carbon atoms and containing single and/or double bonds.

Another embodiment of the invention is a method for enhancing a perfume by incorporating an olfactory acceptable amount of the compounds provided above.

These and other embodiments of the present invention will be apparent by reading the following specification.

### DETAILED DESCRIPTION OF THE INVENTION

In Formula I above, R, R1 and R2 represent a hydrogen or a straight, branched, or cyclic hydrocarbon moiety consisting of 1 to 30 carbon atoms and containing single and/or double bonds. Suitable straight hydrocarbon moieties include ethyl, propyl, butyl, pentyl, hexyl, and the like. Suitable branched hydrocarbon moieties include isopropyl, sec-butyl, tert-butyl, 2-ethyl-propyl, and the like. Suitable cyclic hydrocarbon moieties include cyclopropane, cyclobutane, cyclopentane, cyclopentene, 1,4-cyclopentene, cyclohexane, cyclohexene and the like. Suitable hydrocarbon moieties containing double bonds include ethene, propene, 1-butene, 2-butene, penta-1 -3-deine, hepta-1,3,5-triene and the like.

In the preferred embodiment of the invention, the novel compounds of the present invention are represented by the following structures:

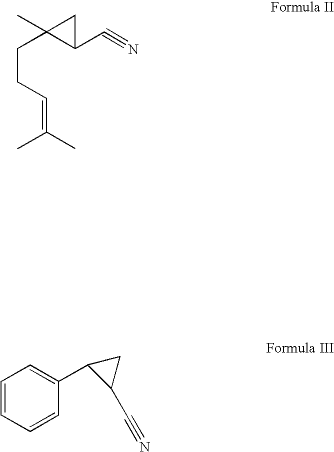

Those with the skill in the art will appreciate that the compound of Formula II is 2-methyl-2-(4-methylpent-3-en-1-yl)-cyclopropanecarbonitrile and the compound of Formula III is 2-phenyl-cyclopropanecarbonitrile.

The table below lists additional compounds derived from Formula I that are described in the present invention:

| R | R1 | R2 | Compound |
|---|---|---|---|
| (CH₂)₂CH₃ | H | H | 2-propyl-cyclopropanecarbonitrile |
| (CH₂)₂CH₃ | CH₃ | H | 2-methyl-2-propyl-cyclopropanecarbonitrile |
| (CH₂)₄CH₃ | H | H | 2-pentyl-cyclopropanecarbonitrile |
| (CH₂)₄CH₃ | CH₃ | H | 2-methyl-2-pentyl-cyclopropanecarbonitrile |
| CHCH(CH₂)₃CH₃ | H | H | 2-hex-1-enyl-cyclopropanecarbonitrile |
| CHCH(CH₂)₃CH₃ | CH₃ | H | 2-methyl-2-hex-1-enyl- |
| | | | cyclopropanecarbonitrile |
| C(CH₃)₃ | H | H | 2-tert-butyl-cyclopropanecarbonitrile |
| C(CH₃)₃ | CH₃ | H | 2-methyl-2-tert-butyl-cyclopropanecarbonitrile |
| (CH₂)₃C(CH₃)₃ | H | H | 2-(4,4-dimethyl-pentyl)- |
| | | | cyclopropanecarbonitrile |
| (CH₂)₃C(CH₃)₃ | CH₃ | H | 2-methyl-2-(4,4-dimethyl-pentyl)- |
| | | | cyclopropanecarbonitrile |
| (CH)₄CH₂CH₃ | H | H | 2-hex-1-dienyl-cyclopropanecarbonitrile |
| (CH)₄CH₂CH₃ | CH₃ | H | 2-methyl-2-hex-1-dienyl- |
| | | | cyclopropanecarbonitrile |
| CH₂C(C₃H₇)(C₄H₉)H | H | H | 2-(2-propyl-hexyl)-cyclopropanecarbonitrile |
| CH₂C(C₃H₇)(C₄H₉)H | CH₃ | H | 2-methyl-2-(2-propyl-hexyl)- |
| | | | cyclopropanecarbonitrile |
| C(CH₃)(CHC(CH₃)₃) | H | H | 2-(1,3,3-trimethyl-but-1-enyl)- |
| | | | cyclopropanecarbonitrile |
| C(CH₃)(CHC(CH₃)₃) | CH₃ | H | 2-methyl-2-(1,3,3-trimethyl-but-1-enyl)- |
| | | | cyclopropanecarbonitrile |
| C₆H₁₁ | H | H | 2-cyclohexyl-cyclopropanecarbonitrile |
| C₆H₁₁ | CH₃ | H | 2-methyl-2-cyclohexyl- |
| | | | cyclopropanecarbonitrile |
| C₆H₉ | H | H | 2-cyclohex-3-enyl-cyclopropanecarbonitrile |
| C₆H₉ | CH₃ | H | 2-methyl-2-cyclohex-3-enyl- |
| | | | cyclopropanecarbonitrile |
| H | H | 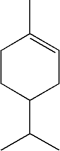 | 1-[1-(4-methyl-3-cyclohexen-1-yl)-ethyl- cyclopropanecarbonitrile |

The compounds of the present invention may be prepared from the corresponding alkenenitriles, via Corey's cyclopropanation reaction. As described in the Examples below, compounds of Formulae II and III may be prepared via Corey's cyclopropanation reaction from the corresponding alkenenitriles below:

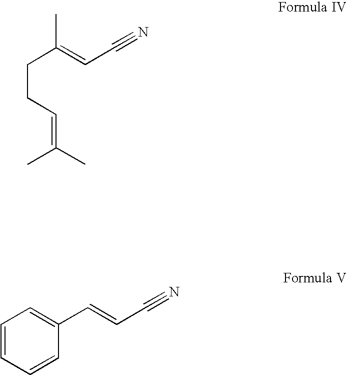

The alkenenitriles of Formulae IV and V are fragrance products commercially available from International Flavors & Fragrances Inc., New York, N.Y. The compound of Formula IV is 3,7-dimethyl-octa-2,6-dienenitrile, which is also known under the tradename Citralva. The compound of Formula V is 2-phenyl-acrylonitrile, which is also known under the tradename Cinnamalva.

Those with skill in the art will recognize that the compounds of the present invention have a number of chiral centers, thereby providing several isomers of the claimed compounds. It is intended herein that the compounds described herein include isomeric mixtures of such compounds, as well as those isomers that may be separated using techniques known to those having skill in the art. Suitable techniques include chromatography such as HPLC, and particularly gel chromatography and solid phase microextraction (“SPME”).

We have discovered that the compounds of Formulae II and III have fresh, citrus, green, cinnamon, musk, sweet, floral tones that are well suited for use as a fragrance ingredient.

The use of the compounds of the present invention is widely applicable in current perfumery products, including the preparation of perfumes and colognes, the perfuming of personal care products such as soaps, shower gels, and hair care products as well as air fresheners and cosmetic preparations. The present invention can also be used to perfume cleaning agents, such as, but not limited to detergents, dishwashing materials, scrubbing, compositions, window cleaners and the like.

In these preparations, the compounds of the present invention can be used alone or in combination with other perfuming compositions, solvents, adjuvants and the like. The nature and variety of the other ingredients that can also be employed are known to those with skill in the art.

Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like.

A list of suitable fragrances is provided in U.S. Pat. No. 4,534,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fem, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

Olfactory effective amount is understood to mean the amount of compound in perfume compositions the individual component will contribute to its particular olfactory characteristics, but the olfactory effect of the perfume composition will be the sum of the effects of each of the perfumes or fragrance ingredients. Thus the compounds of the invention can be used to alter the aroma characteristics of the perfume composition, or by modifying the olfactory reaction contributed by another ingredient in the composition. The amount will vary depending on many factors including other ingredients, their relative amounts and the effect that is desired.

The level of compound of the invention employed in the perfumed article varies from about 0.005 to about 10 weight percent, preferably from about 0.5 to about 8 and most preferably from about 1 to about 7 weight percent. In addition to the compounds other agents can be used in conjunction with the fragrance. Well known materials such as surfactants, emulsifiers, polymers to encapsulate the fragrance can also be employed without departing from the scope of the present invention.

Another method of reporting the level of the compounds of the invention in the perfumed composition, i.e., the compounds as a weight percentage of the materials added to impart the desired fragrance. The compounds of the invention can range widely from 0.005 to about 70 weight percent of the perfumed composition, preferably from about 0.1 to about 50 and most preferably from about 0.2 to about 25 weight percent. Those with skill in the art will be able to employ the desired level of the compounds of the invention to provide the desired fragrance and intensity.

The following are provided as specific embodiments of the present invention. Other modifications of this invention will be readily apparent to those skilled in the art. Such modifications are understood to be within the scope of this invention. As used herein all percentages are weight percent unless otherwise noted, ppm is understood to stand for parts per million and g is understood to be grams. IFF as used in the examples is understood to mean International Flavors & Fragrances Inc.

### EXAMPLE A

### Preparation of 2-methyl-2-(4-methylpent-3-en-1-yl)-cyclopropanecarbonitrile

To a dry 2 L multi-neck round bottom flask fitted with an air stirrer, nitrogen inlet condenser and an addition funnel, 22 g of 60% sodium hydride (NaH), 50 ml of hexane and 400 ml of dimethyl sufoxide (DMSO) was added and stirred. To the resulting mixture 124 g of trimethyloxosulphonioum iodide ((CH₃)₃SOI) was added slowly. After the addition of (CH₃)₃SOI, 75 g of 3,7-dimethyl-octa-2,6-dienenitrile was added dropwise. The mixture was heated to 40° C. and maintained at this temperature for 5 hours. The mixture was aged overnight. In the morning to the mixture was added 200 ml of saturated NH₄Cl, followed by 200 ml of methyl butyl ether. The mixture was shaken, allowed to settle and the lower layer was removed. The gas chromatography test indicated that 77.2% of the starting material converted into 2-methyl-2-(4-methylpent-3-en-1-yl)-cyclopropanecarbonitrile.

The NMR spectrum of the 2-methyl-2-(4-methylpent-3-en-1-yl)-cyclopropanecarbaldehyde is as follows: 0.9-1.0 ppm (m, 2H); 1.1 ppm (s, 1H); 1.2 ppm (m, 1H); 1.2-1.4 ppm (m, 3H); 1.5 ppm (m, 1H); 1.6 ppm (m, 1H); 1.6 ppm (m, 3H); 1.7 ppm (m, 3H); 2.1 ppm (m, 1H); 2.2 ppm (s, 1H); 5.1 ppm (d, 1H).

### EXAMPLE B

### Preparation of 2-phenyl-cyclopropanecarbonitrile

To a dry 500 ml multi-neck round bottom flask fitted with an air stirrer, nitrogen inlet condenser and an addition funnel, 5 g of 60% sodium hydride (NaH), 10 ml of hexane, 100 ml of dimethyl sufoxide (DMSO) and 25 g of trimethyloxosulphonioum iodide ((CH₃)₃SOI) was added and stirred. To the resulting mixture there was added 12.9 g of 2-phenyl-acrylonitrile. The mixture was aged overnight. In the morning to the mixture was added 100 ml of methyl butyl ether, the mixture was quenched with 250 ml of water, stirred and allowed to settle. The organic layer was separated. To the aqueous layer 50 ml of methyl butyl ether was added, the mixture was stirred and allowed to settle. The organic layer was separated. The organic layers were combined and washed with 2×200 ml portions of brine. The gas chromatography test indicated that 59.6% of the starting material converted into 2-phenyl-cyclopropanecarbonitrile.

The NMR spectrum of the 2-phenyl-cyclopropanecarbonitrile is as follows: 1.4 ppm (m, 1H); 1.5 ppm (m, 1H); 1.6 ppm (m, 1H); 2.7 ppm (m, 1H); 7.1 ppm (s, 2H); 7.3 ppm (d, 2H).

### EXAMPLE C

### Incorporation of 2-methyl-2-(4-methylpent-3-en-1-yl)-cyclopropanecarbonitrile—Cyclopropanated Citralva into a fragrance formulation

| | |
|---|---|
| Allyl Cyclohexyl Propionate | 6 |
| Ambroxan | 1 |
| Cyclopropanated Citralva | 115 |
| Citronellyl Nitrile | 130 |
| Cyclabute | 25 |
| Cyclacet | 64 |
| Dihydro Myrcenol | 192 |
| Hexyl Cinnamic Aldehyde | 96 |
| Hexyl Salicylate | 64 |
| Iso E Super | 128 |
| Methyl Anthranilate | 77 |
| Triplal | 6 |
| Vertenex | 96 |
| Total weight | 1000 |

What is claimed is:

1. A compound of formula 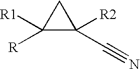
wherein R is a straight, branched, or cyclic hydrocarbon moiety consisting of 1 to 30 carbon atoms and containing single and/or double bonds and R1 is a hydrogen or a straight, branched, or cyclic hydrocarbon moiety consisting of 1 to 30 carbon atoms and containing single and/or double bonds.

2. A method of improving, enhancing or modifying a fragrance formulation through the addition of an olfactory acceptable amount of the compound of claim 1.

3. The method of claim 2 wherein the fragrance is incorporated into a product selected from perfumes, colognes, toilet waters, cosmetic products, personal care products, fabric care products, cleaning products and air fresheners.

4. The method of claim 3 wherein the cleaning product is selected from the group consisting of detergents, dishwashing compositions, scrubbing compounds and window cleaners.

5. The method of claim 2, wherein the amount incorporated into a fragrance is from about 0.005 to about 10 weight percent.

6. The method of claim 2, wherein the amount incorporated into a fragrance is from about 0.5 to about 8 weight percent.

7. The method of claim 2, wherein the amount of incorporated into a fragrance is from about 1 to about 7 weight percent.

8. A fragrance formulation containing an olfactory effective amount of the compound of claim 1.

9. A fragrance product containing a compound of claim 1.
